SEQUENCE LISTING (2 sequences)

SEQ ID NO: 1 - protein, 9 aa, Artificial Seuqence
  Synthetic
  MISC_FEATURE (1)..(1): Pyroglutamate
DGNEEMGGC

SEQ ID NO: 2 - protein, 27 aa, Artificial Sequence
  Synthetic
QDGNEEMGGITQTPYKVSISGTTVILT

(19) United States
(12) Patent Application Publication
(10) Pub. No.: US 2024/0052300 A1
(43) Pub. Date: Feb. 15, 2024

(54) DIFFUSION GRADIENT ASSAY FOR ANTI-CD3-CONTAINING MOLECULES

(71) Applicants: AMGEN RESEARCH (MUNCH) GMBH, Munich (DE); AMGEN INC., Thousand Oaks, CA (US)

(72) Inventors: Glenn Christopher Tan, Thousand Oaks, CA (US); Ewelina Zasadzinska, Thousand Oaks, CA (US); Patrick Hoffmann, Munich (DE); Bin Wu, Thousand Oaks, CA (US)

(21) Appl. No.: 18/266,689

(22) PCT Filed: Dec. 21, 2021
(86) PCT No.: PCT/US21/64546

§ 371 Date: Jun. 12, 2023

Related U.S. Application Data
(60) Provisional application No. 63/128,959, filed on Dec. 22, 2020.

(51) Int. Cl. C12N 5/00; C12M 3/06

(52) U.S. Cl. CPC C12N 5/0075; C12M 23/16

(57) ABSTRACT

The present disclosure provides materials and methods identifying, selecting, and characterizing cells that express and secrete non-Fc containing biomolecules.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “55333_Seqlisting.txt”, which was created on Dec. 7, 2021 and is 835 bytes in size. The subject matter of the Sequence Listing is incorporated herein in its entirety by reference.

### BACKGROUND

The increasing number of candidate therapeutic biologics currently under development has boosted the demand for innovative solutions from Process Development operations to deliver these medicines. A substantial number of biologics are manufactured using live cell systems, where mammalian Chinese Hamster Ovary (CHO) cells are predominantly used for production of recombinant protein therapeutics, such as monoclonal antibodies or other antibody formats (Wurm, F. M., Nat Biotechnol 2004, 22 (11), 1393-8). A typical mammalian Cell Line Development (CLD) process begins with host cell transfection with DNA constructs encoding a transgene of interest, followed by selection and amplification (if necessary) to deliver stable pools of cells expressing desired therapeutic proteins. To meet regulatory requirements and ensure safe and robust process for therapeutic protein production, the process cell lines must be derived from a single cell (Frye, C.; et al., Biologicals 2016, 44 (2), 117-22; and Le K, Tan C, Le H, et al. Assuring Clonality on the Beacon Digital Cell Line Development Platform. Biotechnol J. 2020; 15(1):e1900247). The pool populations of cells used for single cell cloning are highly heterogeneous, where cells producing high amounts of protein with satisfying product quality profiles are poorly represented. Therefore, empirical screening efforts need to be employed to identify and isolate highly quality clonal cell lines with desired product quality attributes. Typically, clone screening studies are considerably challenging and resource intensive as hundreds to thousands of clones need to be evaluated in order to identify high quality candidate cell lines suitable for clinical and commercial manufacturing (Wurm, F. M., Nat Biotechnol.

The recently developed Berkeley Light's Beacon® technology platform is suitable for cell line development operations and enables assessment of growth and desired secretory profiles at the single cell level. The Beacon® instrument is a fully integrated nanofluidic cell culture system that allows to isolate up to 1758 clonal cell lines on a single nanofluidic chip. The BLI platform is equipped with a built-in fluorescence microscopy capabilities, enabling development of fluorescent assays at nanoscale to characterize cell populations grown on chip. Cells loaded into the nanofluidic chip are isolated in individual pens and can be simultaneously cultured and assayed for recombinant protein secretion. Candidate clones are selected based on desired growth and secretory profiles, exported off the chip and subjected to scale-up. The Beacon® platform was recently used to enrich for high quality clones suitable for clinical and commercial manufacturing with high assurance of clonality (Le K, et al., Biotechnol Prog. 2018; 34(6):1438-1446; and Le K, et al., Biotechnol J. 2020; 15(1):e1900247).

A standard CLD workflow utilizing the BLI platform allows for selection of highly producing cell lines expressing therapeutic biologics with human Fc or human Kappa Light Chain domains present in their protein sequence. Screening for cell lines exhibiting desired secretory phenotypes can be facilitated through BLI's diffusion based Spotlight™ Hu3 or SpotLight Human Kappa Assays that allow for protein detection on a nanofluidic chip and to assess secretion capacity of individual clones. Berkeley Light's diffusion assay reagents contains fluorescently labelled probe that binds to human Fc or human Kappa Light chain domains present in subset of therapeutic modalities such as monoclonal antibodies. The assay relies on differential retention of fluorescent reagent in pens, which can be detected by fluorescent microscopy and provides a quantitative measure of recombinant protein secretion. The small molecular weight of the fluorescent probe allows for its rapid diffusion and efficient on-chip equilibration. The assay reagent is diluted in medium and perfused on chip where it diffuses into each pen and binds to target domain present in secreted protein product. As a consequence, a high molecular weight complex consisting of the target protein and diffusion assay reagent is formed. When the equilibrium is reached, the chip is flushed with medium, and during this process the unbound assay reagent diffuses out of pens rapidly. In contrast, the diffusion reagent complexed with secreted protein is retained in pens due to its slower diffusion rate caused by its larger molecular weight. Fluorescent intensity corresponding to Spotlight™ Hu3-Fc domain or SpotLight Human Kappa-Kappa Light chain complexes is detected in the pen and allows to measure recombinant protein secretion levels.

A significant limitation to utilizing Berkeley Lights diffusion assays for cell line screening is that they can only be applied to cells expressing subset of recombinant proteins, such as antibodies or other human Fc or human Kappa Light Chain containing antibody formats. There is a substantial amount of recombinant protein therapeutics however, that may not be compatible with commercially available reagents provided by Berkeley Lights due to a molecule format or protein sequence engineering. Bi-specific T cell engager (BiTE®) molecules are recombinant fusion proteins consisting of single-chain variable fragments (scFv) of two antibodies and are engineered to specifically recognize two antigens: one present on a T cell surface through affinity to the CD3 receptor, and a second antigen found on a target tumor cell (Baeuerle P A, et al., Curr Opin Mol Ther. 2009; 11(1):22-30; Frankel S R, and Baeuerle P A. Curr Opin Chem Biol. 2013; 17(3):385-392; and Yuraszeck T, et al., Clin Pharmacol Ther. 2017; 101(5):634-645). BiTE® therapy is an anti-cancer treatment designed to engage patient's own immune system to combat the disease. Upon BiTE® administration, the patient's own T cells are tethered to cancer cells expressing a selected tumor-associated antigen, and consequently induce an immune response against the tumor. BiTE® technology offers a promising solution in developing treatments against both solid and hematologic malignancies (Baeuerle P A, et al., Curr Opin Mol Ther. 2009; 11(1):22-30).

Thus, there exists a need in the art to develop methods of characterizing cells that express and secrete non-Fc containing biomolecules such as canonical BiTE® molecules.

### SUMMARY OF THE INVENTION

As described herein, the present disclosure provides, in various embodiments, methods for isolating at least one cell from a population of cells that secretes a biomolecule capable of binding a CD3 peptide. In one embodiment, the present disclosure provides a method for isolating at least one cell from a population of cells that secretes a biomolecule capable of binding a CD3 peptide comprising the steps of: (a) collecting a single cell from a population of cells in a nanofluidic chamber of a nanofluidic chip, wherein said cell comprises an expression construct capable of expressing said biomolecule; (b) culturing the single cell under conditions that allow clonal expansion and expression and secretion of said biomolecule, thereby producing multiple cells from a single cell clone; (c) administering a composition comprising said CD3 peptide to said nanofluidic chip under conditions that allow the CD3 peptide to contact said biomolecule secreted by said multiple cells of the single cell clone; (d) detecting binding of CD3 peptide and said biomolecule; and (e) isolating at least one cell from the multiple cells of step (b) that secretes a biomolecule capable of binding a CD3 peptide.

In one embodiment, the population of cells is a cell line. In another embodiment, the population of cells is a mixture of two or more cell lines. In still another embodiment, an aforementioned method is provided further comprising the step of quantifying the amount of said biomolecule secreted by said multiple cells of a single cell line.

In still another embodiment, the present disclosure provides an aforementioned method wherein the cell is selected from the group consisting of a mammalian cell, an insect cell, a bacterial cell, a eukaryotic cell, a plant cell, a yeast cell and a fungal cell. In some embodiments, the cell is selected from the group consisting of a Chinese hamster ovary (CHO) cell, human embryonic kidney (HEK) cell, murine myeloma (NS0, Sp2/0) cell, baby hamster kidney (BHK) cell, human embryonic kidney (293) cell, fibrosarcoma (HT-1080) cell, human embryonic retinal (PER.C6) cell, hybrid kidney and B cell (HKB-11), CEVEC's amniocyte production (CAP) cells, and human liver (HuH-7) cell.

In another embodiment, the present disclosure provides an aforementioned method wherein said biomolecule comprises a polypeptide. In one embodiment, the polypeptide is recombinant. In some embodiments, the polypeptide is selected from the group consisting of an antibody, a peptibody, a multispecific protein, a bispecific protein, a bi-specific T cell engager, a half-life extended bi-specific T cell engager, and biologically active fragments, analogs and derivatives thereof.

In still another embodiment, the present disclosure provides an aforementioned method wherein the biomolecule is a BiTE and is also capable of binding a target molecule selected from the group consisting of CD33, EGFRvIII, MSLN, CDH19, DLL3, CD19, FLT3, CDH3, BCMA, PSMA, MUC17, CLDN18.2, EpCAM, CEA, Her2, CD20 and CD70.

In yet another embodiment, the present disclosure provides an aforementioned method wherein said CD3 peptide comprises 5, 6, 7, 8, 9, 10, 11, 12, 13, 14, 15, 16, 17, 18, 19 or 20 amino acids. In one embodiment, the CD3 peptide comprises 10 amino acids. In another embodiment, the CD3 peptide comprises the amino acid sequence Pyroglutamate-DGNEEMGGC (SEQ ID NO: 1).

In still another embodiment, the present disclosure provides an aforementioned method wherein said CD3 peptide is a CD3 peptide-conjugate comprising at least one modification. In one embodiment, the modification comprises attachment of a detection moiety selected wherein said detection moiety is a fluorophore. In one embodiment, the fluorophore is selected from the group consisting of AF594 and AF488. In still another embodiment, the AF594 is attached to the C-terminal cysteine of the sequence Pyroglutamate-DGNEEMGGC (SEQ ID NO: 1) using a maleimide linker.

In yet another embodiment, the present disclosure provides an aforementioned method wherein said expression construct is selected from the group consisting of a vector, a plasmid, and a linearized DNA expression sequence. In still other embodiment, an aforementioned method is completed in 14 days or less.

In another embodiment, the present disclosure provides an aforementioned method wherein said nanofluidic chip comprises between 1,000 and 2,000 nanofluidic chambers. In one embodiment, the nanofluidic chip comprises 1758 nanofluidic chambers.

In still another embodiment, the present disclosure provides a method for isolating at least one cell from a population of cells that secretes a bi-specific T cell engager capable of binding a CD3 peptide comprising the steps of: (a) collecting a single cell in a nanofluidic chamber of a nanofluidic chip, wherein said cell comprises an expression construct capable of expressing said bi-specific T cell engager; (b) culturing the single cell under conditions that allow clonal expansion and expression and secretion of said bi-specific T cell engager, thereby producing multiple cells from a single cell clone; (c) administering a composition comprising said CD3 peptide to said nanofluidic chip under conditions that allow the CD3 peptide to contact said bi-specific T cell engager secreted by said multiple cells of the single cell line; (d) detecting binding of CD3 peptide and said bi-specific T cell engager; and (e) isolating at least one cell from the multiple cells of step (b) that secretes a bi-specific T cell engager capable of binding a CD3 peptide; wherein said CD3 peptide is a CD3 peptide conjugate and comprises the amino acid sequence Pyroglutamate-DGNEEMGGC (SEQ ID NO: 1), wherein AF594 is attached to the C-terminal cysteine of said sequence.

In yet another embodiment, the present disclosure provides a method of producing a biomolecule capable of binding a CD3 peptide comprising the steps of: (a) collecting a single cell from a population of cells in a nanofluidic chamber of a nanofluidic chip, wherein said cell comprises an expression construct capable of expressing said biomolecule; (b) culturing the single cell under conditions that allow clonal expansion and expression and secretion of said biomolecule, thereby producing multiple cells from a single cell clone; (c) administering a composition comprising said CD3 peptide to said nanofluidic chip under conditions that allow the CD3 peptide to contact said biomolecule secreted by said multiple cells of the single cell line; (d) detecting binding of CD3 peptide and said biomolecule; (e) isolating at least one cell from the multiple cells of step (b) that secretes a biomolecule capable of binding a CD3 peptide; and (f) transferring said at least one cell from the cell line to a vessel and culturing said cell under conditions that allow production of the biomolecule.

In still another embodiment, the present disclosure provides a method for isolating at least one cell from a population of cells that secretes a biomolecule capable of binding a CD3 peptide comprising the steps of: (a) collecting a single cell from a population of cells in a nanofluidic chamber of a nanofluidic chip, wherein said cell comprises an expression construct capable of expressing said biomolecule; (b) culturing the single cell under conditions that allow clonal expansion and expression and secretion of said biomolecule, thereby producing multiple cells from a single cell clone; (c) administering a first composition comprising said CD3 peptide and a second composition comprising a biomolecule binding-reagent to said nanofluidic chip under conditions that allow the CD3 peptide to contact said biomolecule secreted by said multiple cells of the single cell line; (d) detecting binding of the CD3 peptide and the biomolecule binding agent and said biomolecule; and (e) isolating at least one cell from the multiple cells of step (b) that secretes a biomolecule capable of binding a CD3 peptide and, optionally, the biomolecule binding agent. In some embodiments, the aforementioned method allows isolation at least one cell from a mixture of cells or cell lines or mixed population of cells or cell lines. In some embodiments, the population of cells are from one cell line.

### BRIEF DESCRIPTION OF THE DRAWINGS

FIG. 1 shows the design and development of the assay reagent mimicking CD3 binding. FIG. 1A. Schematic representation of BiTE® specific reagent design for diffusion gradient assay. FIG. 1B. SEC profile corresponding recombinant BiTE® protein purified from medium supplemented with CD3-AF488 peptide conjugate. Detected species of recombinant BiTE® protein are depicted by arrows. FIG. 1C. SEC profile corresponding to a sample derived from secreted medium supplemented with CD3-AF488 peptide conjugate. Detected species of recombinant BiTE® protein and unbound CD3-AF488 regent are depicted by arrows. FIG. 1D. Graph demonstrating the viability of CHO cells measured after 24 h incubation with CD3 diffusion assay reagent at indicated concentrations.

FIG. 2 shows the optimization of diffusion gradient assay for CD3 binder secretion on Berkeley Lights nanofluidic chip. FIG. 2A. Schematic representation of on-chip CD3 diffusion gradient assay to detect secretion of a CD3 binder protein, a canonical BiTE® protein product.

FIG. 2B. Images acquired on BLI platform demonstrating the CD3 diffusion assay allows identification clones secreting a canonical BiTE® modality based on differential retention of a fluorescent probe in pens.

FIG. 3 shows the CD3 diffusion assay demonstrates high specificity towards two BiTE® formats on Berkeley Lights nanofluidic chip. FIG. 3A. Representative images acquired on BLI platform demonstrating the specificity and efficiency of CD3 binder diffusion assay. The images were extracted from the Assay Analyzer tool built in the BLI platform. The exposure time used for CD3 binder and Spotlight® Hu3 assays image acquisition are not the same due to differences in assay parameters. Three independently generated cell lines expressing a canonical BiTE®, a BiTE®-Fc fusion or an IgG fusion protein were loaded in alternating sections on one chip and cultured for the duration of 5 days. The CD3 binder and Spotlight® Hu3 assays were executed on day 5. FIG. 3B. Graphs demonstrating the quantification of secretion score and assay results shown in A. FIG. 3C. Correlation of CD3 diffusion assay vs Spotlight® Hu3 assay scores for three modalities expressed by cell lines shown in A and B. The R² values displayed demonstrate the degree of correlation between the CD3 diffusion assay and Spotlight® Hu3 assay results.

FIG. 4 shows clones selected based on the CD3 diffusion assay exhibit similar performance compared to parental cell line used for subcloning. FIG. 4A, FIG. 4B. Graphs demonstrating normalized titer measurements (A) and specific productivity (B) assessed by a 10 day fed batch experiments for BiTE®-Fc fusion expressing clones that were selected based on CD3 diffusion assay and exported off the chip shown in FIG. 3. BLI exported clones generated in this study are shown with circles and parental cell line used for subcloning is shown with triangles. The titer and Qp achieved by cell lines shown in A and B were normalized to values measured for parental cell line. FIG. 4B. Graphs demonstrating viable cell density and viability during the 10 day fed batch experiments shown in A and B.

FIG. 5 shows the development of dual targeting diffusion gradient assay for BiTE® modalities secreted at low levels. FIG. 5A. Schematic representation of experimental designed shown in B and C. FIG. 5B. Graphs demonstrating the quantification of CD3 diffusion assay, Spotlight® Hu3 and dual targeting assay results. BiTE®-Fc expressing cell lines are shown in dark gray circles and empty pens are shown in light gray circles. The secretion score (Au score) was normalized to an average Au score calculated for empty pens within each dataset. FIG. 5C. Representative images acquired on BLI platform demonstrating the efficiency of dual targeting diffusion gradient assay. The exposure time used for CD3 diffusion assay/dual targeting assay and Spotlight® Hu3 assay image acquisition are not the same due to differences in assay parameters.

### DETAILED DESCRIPTION

The present disclosure addresses the aforementioned need in the art by providing methods and materials useful for characterizing cells that express and secrete non-Fc containing biomolecules. Further enhancing this need, both canonical and half-life extended Fc-conjugated BiTE® molecules are both expressed at lower levels than monoclonal antibodies in CHO cells. A screening method to identify clones expressing high levels of BiTE® molecules potentially bypasses the hurdle of low-expressing pools.

In one embodiment, the present disclosure provides an assay specific for CD3 binding modalities, that is compatible with cell line development workflow using the Beacon® platform. For this embodiment, a CD3-peptide conjugate was prepared and an optimized on-chip diffusion gradient assay was performed in order to identify clones secreting CD3 binding therapeutic biologics on a nanofluidic chip. This strategy allows clones secreting high levels of CD3 binders to be distinguished from low producing clones, and from cell lines secreting other modalities on a nanofluidic chip. Furthermore, cell lines selected using this assay demonstrate similar growth and secretion profiles when compared to clones derived via standard workflows. The CD3 diffusion assay provided herein thus allows for the identification of high secreting cell lines on a nanofluidic chip and supports early clone selection using, in one embodiment, a BLI platform. In other embodiments, the present disclosure provides methods and reagents that can be used in other microfluidic devices, cell-based microarrays, microtiter plate-based screening assays (e.g., ELISA) and droplet-based screens using bead encapsulation techniques, including lithographic-based microarrays and nanowell-assisted cell patterning platforms (Love et al., Nat. Biotechnol., 24(6) 703 (2006); and Ozkumur et al., Materials Views, 11(36), 4643-4650 (2015).

Cell line development campaigns involve thousands of single progenitor cell lines that are expanded and evaluated for secretion of desired therapeutic biologics. The Berkeley Light Beacon® platform is a fully integrated nanofluidic cell culture system that enables the user to simultaneously culture, assay and grow up to 1758 clonal cell lines on a single nanofluidic chip. As described herein, in one embodiment, the Beacon® platform advances cell line development (CLD) operations and enables assessment of growth and desired secretory profiles at the single cell level. This allows selection of high-quality clones without the need to scale up thousands of candidates to production scale and screening using standard assays. For on-chip secretion assessment Berkeley Lights developed the Spotlight™ Hu3 and Spotlight Human Kappa assay reagents that bind to human Fc or human Kappa Light Chain domains respectively, however, this approach only applies to a subset of recombinant protein therapeutics. The present disclosure thus provides, in one embodiment, an on-chip diffusion-based assay that utilizes fluorophore conjugated peptides specifically recognized by CD3 binders, such as canonical bispecific T-cell engagers, enabling identification of highly secreting clones on a nanofluidic chip. As disclosed herein, the assay provides specificity for detecting various CD3 binders. Furthermore, in another embodiment, the present disclosure provides a dual targeting assay strategy using two fluorescent probes to detect recombinant protein secretion and enabling increased sensitivity of standard diffusion gradient assays.

The present disclosure provides, in one embodiment, a method for isolating at least one cell from a population of cells that secretes a biomolecule capable of binding a CD3 peptide. The phrase “at least 1” as used herein can be 1, 2, 3, 4, 5, 6, 7, 8, 9, 10 or more. In some embodiments, thousands of individual cells are isolated and assayed. For example, in some embodiments approximately 10, 100, 1,000, 5,000, 6,000, 7,000, 8,000, 9,000, 10,000 or 11,000 or more cells are isolated. In some embodiments, nanofluidic chips can accommodate ever higher numbers of individual cells to be isolated, up to and including, for example, 85,000 or 250,0000 individual cells. In one embodiment, 1758 cells are isolated on a nanofluidic chip. The “population of cells” in one embodiment is a population of cells of a single cell line. The single cell line can be cloned, in one embodiment of the present disclosure. In other embodiments, the population of cells is from a mixture (e.g., 2 or more) of cell lines (e.g., a mixed population of cells).

The biomolecules provided herein are capable of binding to a CD3 protein or a CD3 peptide. In one embodiment, the CD3 protein and the CD3 peptides described herein correspond to UniProt No. P07766. (See also Clevers et al., Proc. Natl. Acad. Sci., 85, 8156-8160 (1988) and Borroto et al., J. Immunol., 163, (1), 25-31 (1999) for additional information and sequences of CD3 contemplated herein). The CD3 peptide may comprise a portion of the endogenous human CD3 epsilon protein sufficient to facilitate binding in the methods described herein. In some embodiments, the CD3 peptide is 5, 6, 7, 8, 9, 10, 11, 12, 13, 14, 15, 16, 17, 18, 19 or 20 or more consecutive amino acids of the full length CD3 amino acid sequence. In some embodiments, the CD3 peptide is 59, 10, 11, 12, 13 or 14 consecutive amino acids of the full length CD3 amino acid sequence. In some embodiments, the CD3 peptide amino acid sequence may be modified (e.g., include a substitution, deletion and/or insertion mutation and/or include a chemical modification). In some embodiments, the CD3 peptide amino acid sequence may be modified to include a carrier protein at one or both termini. As described herein, the peptide can optionally be synthetically conjugated to pyroglutamate. In another embodiment, one or more cysteine (C) residues may be added, either internally or at one or more peptide terminus, to facilitate chemical modification. By way of example, the CD3 peptide comprises the sequence DGNEEMGGC (SEQ ID NO: 1). In some embodiments, the addition of a C-terminal cysteine and/or fluorophore moiety improves solubility of the peptide. For example, while an unmodified peptide alone can be insoluble, addition of a cysteine can allow for fluorophore conjugation and increase solubility.

Thus, as described herein the CD3 peptide may comprise a modification to the following sequence or portion of the following sequence (the first 27 N-terminal amino acids of CDR), including but not limited to one or more substitution, deletion and/or insertion mutations and/or inclusion of a tag or a chemical modification: QDGNE EMGGI TQTPY KVSIS GTTVI LT (SEQ ID NO: 2).

Conjugation of reporter molecules such as fluorophores to the CD3 peptide are also contemplated by the present disclosure. In some embodiments, the fluorophore is charged or highly charged to achieve solubility of the conjugated peptide. In some embodiments, the fluorophores have carboxy groups exchanged with sulfate groups and thus have higher solubility at even low pH. In some embodiments, Alexa Fluor® Dyes or Amersham™ CyDye™ fluors or a VivoTag® fluorochrome (Perkin Elmer) or a DyLight® fluorochrome are used in the methods described herein. By way of example, the peptide can be conjugated to an ALEXA-FLUOR® dye (ThermoFisher Scientific) such as ALEXA-FLUOR® 488, ALEXA-FLUOR® 594, ALEXA-FLUOR® 555, ALEXA-FLUOR® 647. In some embodiments, ALEXA-FLUOR® 350, ALEXA-FLUOR® 405, ALEXA-FLUOR® 532, ALEXA-FLUOR® 546, ALEXA-FLUOR® 568, ALEXA-FLUOR®, ALEXA-FLUOR® 700, ALEXA-FLUOR® 750, BODIPY FL, Coumarin, Cy3, Cy5, Cy2, Cy3.5, Cy5.5, Cy7, Fluorescein (FITC), Oregon Green, Pacific Blue, Pacific Green, Pacific Orange, PE-Cyanine7, PerCP-Cyanine5.5, Tetramethylrhodamine (TRITC), and/or Texas Red, each available from ThermoFischer Scientific, are contemplated herein.

In some embodiments, ALEXA-FLUOR® 488 or ALEXA-FLUOR® 594 are conjugated to the peptide via maleimide mediated chemistry.

In other embodiments, a peptide according to the present disclosure may be modified using one or more of the following exemplary techniques: click chemistry, direct conjugation via inclusion of a chemically-activated fluorophore during peptide synthesis, covalent coupling of fluorophore to an artificial amino acid used during peptide synthesis, and/or adding an N-terminal lysine.

As described herein, in one embodiment the compositions and methods provided by the present disclosure enable the identification of biomolecules capable of binding to a CD3 protein or a CD3 peptide. In related embodiments, the biomolecule is a bispecific biomolecule capable of binding a CD3 protein or a CD3 peptide and an antigen target as described herein.

As used herein, the term “biomolecule” refers to a molecule produced by a cell that is capable of binding to a binding reagent such as the CD3 peptide described herein. A biomolecule, according to various embodiments of the disclosure, includes but is not limited to, an antibody, a peptibody, a fusion protein, a mutein, a multispecific protein, a bispecific protein, as well as biologically active fragments, analogs, derivatives and variants, as well as biosimilars, thereof.

“Multispecific protein” and “multispecific antibody” are used herein to refer to proteins that are recombinantly engineered to simultaneously bind, neutralize and/or interact specifically with at least two different antigens or at least two different epitopes on the same antigen. For example, multispecific proteins may be engineered to target immune effectors in combination with targeting cytotoxic agents to tumors or infectious agents. Multispecific proteins that bind two antigens, referred to herein as “bispecific proteins”, and “bispecific antibodies”, are the most common and diverse group of multispecific proteins. The formats for bispecific proteins, which include bispecific antibodies, include, but are not limited to, quadromas, knobs-in-holes, cross-Mabs, dual variable domains IgG (DVD-IgG), IgG-single chain Fv (scFv), scFv-CH3 KIH, dual action Fab (DAF), half-molecule exchange, κλ-bodies, tandem scFv, scFv-Fc, diabodies, single chain diabodies (scDiabodies), scDiabodies-CH3, triple body, miniantibody, minibody, TriBi minibody, tandem diabodies, scDiabody-HAS, Tandem scFv-toxin, dual-affinity retargeting molecules (DARTs), nanobody, nanobody-HSA, dock and lock (DNL), strand exchange engineered domain SEEDbody, Triomab, leucine zipper (LUZ-Y), Fab-arm exchange, DutaMab, DT-IgG, charged pair, Fcab, orthogonal Fab, IgG(H)-scFv, scFV-(H)IgG, IgG(L)-scFV, IgG(L1H1)-Fv, IgG(H)-V, V(H)—IgG, IgG(L)-V V(L)-IgG, KIH IgG-scFab, 2scFV-IgG, IgG-2scFv, scFv4-Ig, Zybody, DVI-Ig4 (four-in-one), Fab-scFv, scFv-CH-CL-scFV, F(ab′)2-scFv2, scFv-KIH, Fab-scFv-Fc, tetravalent HCAb, scDiabody-Fc, diabody-Fc, intrabody, ImmTAC, HSABody, IgG-IgG, Cov-X-Body, scFv1-PEG-scFv2, a molecule made using XmAb® technology, bi-specific T cell engagers (BiTE®), and half-life extended bispecific T cell engagers (HLE BiTE®) (Fan supra; Spiess supra; Sedykh supra; Seimetz et al., Cancer Treat Rev 36(6) 458-67, 2010; Shulka and Norman, Chapter 26 Downstream Processing of Fc Fusion Proteins, Bispecific Antibodies, and Antibody-Drug Conjugates, in Process Scale Purification of Antibodies Second Edition, Uwe Gottswchalk editor, p 559-594, John Wiley & Sons, 2017; Moore et al., MAbs 3:6, 546-557, 2011).

Multispecific proteins also include trispecific antibodies, tetravalent bispecific antibodies, multispecific proteins without antibody components such as dia-, tria- or tetrabodies, minibodies, and single chain proteins capable of binding multiple targets, and the like. Coloma, M. J., et. al., Nature Biotech. 15 (1997) 159-163.

In some embodiments, bispecific proteins may include blinatumomab, catumaxomab, ertumaxomab, solitomab, targomiRs, lutikizumab (ABT981), vanucizumab (RG7221), remtolumab (ABT122), ozoralixumab (ATN103), floteuzmab (MGD006), pasotuxizumab (AMG112, MT112), lymphomun (FBTA05), (ATN-103), AMG211 (MT111, Medi-1565), AMG330, AMG420 (B1836909), AMG-110 (MT110), MDX-447, TF2, rM28, HER2Bi-aATC, GD2Bi-aATC, MGD006, MGD007, MGD009, MGD010, MGD011 (JNJ64052781), IMCgp100, indium-labeled IMP-205, xm734, LY3164530, OMP-305BB3, REGN1979, COV322, ABT112, ABT165, RG-6013 (ACE910), RG7597 (MEDH7945A), RG7802, RG7813(RO6895882), RG7386, BITS7201A (RG7990), RG7716, BFKF8488A (RG7992), MCLA-128, MM-111, MM141, MOR209/ES414, MSB0010841, ALX-0061, ALX0761, ALX0141; B11034020, AFM13, AFM11, SAR156597, FBTA05, PF06671008, GSK2434735, MEDI3902, MEDI0700, MEDI7352, as well as the molecules or variants or analogs thereof and biosimilars of any of the foregoing.

As used herein “biologically active derivative” or “biologically active variant” includes any derivative or variant of a molecule having substantially the same functional and/or biological properties of said molecule, such as binding properties, and/or the same structural basis, such as a peptidic backbone or a basic polymeric unit.

An “analog,” such as a “variant” or a “derivative,” is a compound substantially similar in structure and having the same biological activity, albeit in certain instances to a differing degree, to a naturally-occurring molecule. For example, a polypeptide variant refers to a polypeptide sharing substantially similar structure and having the same biological activity as a reference polypeptide. Variants or analogs differ in the composition of their amino acid sequences compared to the naturally-occurring polypeptide from which the analog is derived, based on one or more mutations involving (i) deletion of one or more amino acid residues at one or more termini of the polypeptide and/or one or more internal regions of the naturally-occurring polypeptide sequence (e.g., fragments), (ii) insertion or addition of one or more amino acids at one or more termini (typically an “addition” or “fusion”) of the polypeptide and/or one or more internal regions (typically an “insertion”) of the naturally-occurring polypeptide sequence or (iii) substitution of one or more amino acids for other amino acids in the naturally-occurring polypeptide sequence. By way of example, a “derivative” is a type of analog and refers to a polypeptide sharing the same or substantially similar structure as a reference polypeptide that has been modified, e.g., chemically.

A variant polypeptide is a type of analog polypeptide and includes insertion variants, wherein one or more amino acid residues are added to a biomolecule amino acid sequence of the invention. Insertions may be located at either or both termini of the protein, and/or may be positioned within internal regions of the therapeutic protein amino acid sequence. Insertion variants, with additional residues at either or both termini, include for example, fusion proteins and proteins including amino acid tags or other amino acid labels. In one aspect, the biomolecule optionally contains an N-terminal Met, especially when the molecule is expressed recombinantly in a bacterial cell such as E. coli. In another aspect, the biomolecule includes histidine tag (His-tag).

In deletion variants, one or more amino acid residues in a biomolecule polypeptide as described herein are removed. Deletions can be effected at one or both termini of the therapeutic protein polypeptide, and/or with removal of one or more residues within the therapeutic protein amino acid sequence. Deletion variants, therefore, include fragments of a therapeutic protein polypeptide sequence.

In substitution variants, one or more amino acid residues of a biomolecule are removed and replaced with alternative residues. In one aspect, the substitutions are conservative in nature and conservative substitutions of this type are well known in the art. Alternatively, the invention embraces substitutions that are also non-conservative. Exemplary conservative substitutions are described in Lehninger, [Biochemistry, 2nd Edition; Worth Publishers, Inc., New York (1975), pp. 71-77] and are set out immediately below.

As used herein, a biomolecule that “specifically binds” is “antigen specific”, is “specific for” antigen target or is “immunoreactive” with an antigen refers to a biomolecule that binds an antigen with greater affinity than other antigens of similar sequence. In one aspect, the a biomolecule or fragments, variants, or derivatives thereof, will bind with a greater affinity to human antigen as compared to its binding affinity to similar antigens of other, i.e., non-human, species, but polypeptide binding agents that recognize and bind orthologs of the target are within the scope of the invention.

The term “epitope” refers to that portion of any molecule capable of being recognized by and bound by a biomolecule. Epitopes usually consist of chemically active surface groupings of molecules, such as, amino acids or carbohydrate side chains, and have specific three-dimensional structural characteristics as well as specific charge characteristics. Epitopes as used herein may be contiguous or non-contiguous.

Biomolecules contemplated herein include full-length proteins, precursors of full-length proteins, biologically active subunits or fragments of full length proteins, as well as biologically active derivatives and variants of any of these forms of therapeutic proteins. Thus, biomolecules include those that (1) have an amino acid sequence that has greater than about 60%, about 65%, about 70%, about 75%, about 80%, about 85%, about 90%, about 91%, about 92%, about 93%, about 94%, about 95%, about 96%, about 97%, about 98% or about 99% or greater amino acid sequence identity, over a region of at least about 25, about 50, about 100, about 200, about 300, about 400, or more amino acids, to a polypeptide encoded by a referenced nucleic acid or an amino acid sequence described herein. According to the present disclosure, the term “recombinant biomolecule” includes any biomolecule obtained via recombinant DNA technology. In certain embodiments, the term encompasses proteins as described herein.

In some embodiments, biomolecules of interest bind, neutralize, and/or interact specifically with a CD3 protein or peptide and one or more other proteins such as HER receptor family proteins, CD proteins, cell adhesion molecules, growth factors, nerve growth factors, fibroblast growth factors, transforming growth factors (TGF), insulin-like growth factors, osteoinductive factors, insulin and insulin-related proteins, coagulation and coagulation-related proteins, colony stimulating factors (CSFs), other blood and serum proteins blood group antigens; receptors, receptor-associated proteins, growth hormones, growth hormone receptors, T-cell receptors; neurotrophic factors, neurotrophins, relaxins, interferons, interleukins, viral antigens, lipoproteins, integrins, rheumatoid factors, immunotoxins, surface membrane proteins, transport proteins, homing receptors, addressins, regulatory proteins, and immunoadhesins.

In some embodiments biomolecules of interest bind, neutralize and/or interact with a CD3 protein and one or more of the following, alone or in any combination: CD proteins including but not limited to CD4, CD5, CD7, CD8, CD19, CD20, CD22, CD25, CD30, CD33, CD34, CD38, CD40, CD70, CD123, CD133, CD138, CD171, and CD174, HER receptor family proteins, including, for instance, HER2, HER3, HER4, and the EGF receptor, EGFRvIII, cell adhesion molecules, for example, LFA-1, Mol, p150,95, VLA-4, ICAM-1, VCAM, and alpha v/beta 3 integrin, growth factors, including but not limited to, for example, vascular endothelial growth factor (“VEGF”); VEGFR2, growth hormone, thyroid stimulating hormone, follicle stimulating hormone, luteinizing hormone, growth hormone releasing factor, parathyroid hormone, mullerian-inhibiting substance, human macrophage inflammatory protein (MIP-1-alpha), erythropoietin (EPO), nerve growth factor, such as NGF-beta, platelet-derived growth factor (PDGF), fibroblast growth factors, including, for instance, aFGF and bFGF, epidermal growth factor (EGF), Cripto, transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors, insulins and insulin-related proteins, including but not limited to insulin, insulin A-chain, insulin B-chain, proinsulin, and insulin-like growth factor binding proteins; (coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, thrombopoietin, and thrombopoietin receptor, colony stimulating factors (CSFs), including the following, among others, M-CSF, GM-CSF, and G-CSF, other blood and serum proteins, including but not limited to albumin, IgE, and blood group antigens, receptors and receptor-associated proteins, including, for example, flk2/flt3 receptor, obesity (OB) receptor, growth hormone receptors, and T-cell receptors; neurotrophic factors, including but not limited to, bone-derived neurotrophic factor (BDNF) and neurotrophin-3, -4, -5, or -6 (NT-3, NT-4, NT-5, or NT-6); relaxin A-chain, relaxin B-chain, and prorelaxin, interferons, including for example, interferon-alpha, -beta, and -gamma, interleukins (ILs), e.g., IL-1 to IL-10, IL-12, IL-15, IL-17, IL-23, IL-12/IL-23, IL-2Ra, IL1-R1, IL-6 receptor, IL-4 receptor and/or IL-13 to the receptor, IL-13RA2, or IL-17 receptor, IL-1RAP,

viral antigens, including but not limited to, an AIDS envelope viral antigen, lipoproteins, calcitonin, glucagon, atrial natriuretic factor, lung surfactant, tumor necrosis factor-alpha and -beta, enkephalinase, BCMA, STEAP1, IgKappa, ROR-1, ERBB2, mesothelin, RANTES (regulated on activation normally T-cell expressed and secreted), mouse gonadotropin-associated peptide, Dnase, FR-alpha, inhibin, and activin, integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, MIC (MIC-a, MIC-B), ULBP 1-6, EPCAM, addressins, regulatory proteins, immunoadhesins, antigen-binding proteins, somatropin, CTGF, CTLA4, eotaxin-1, MUC1, CEA, c-MET, Claudin-18, GPC-3, EPHA2, FPA, LMP1, MG7, NY-ESO-1, PSCA, ganglioside GD2, glanglioside GM2, BAFF, OPGL (RANKL), myostatin, Dickkopf-1 (DKK-1), Ang2, NGF, IGF-1 receptor, hepatocyte growth factor (HGF), TRAIL-R2, c-Kit, B7RP-1, PSMA, NKG2D-1, programmed cell death protein 1 and ligand, PD1 and PDL1, mannose receptor/hCG3, TNF, TL1A, hepatitis-C virus, mesothelin dsFv[PE38 conjugate, Legionella pneumophila (lly), IFN gamma, interferon gamma induced protein 10 (IP10), IFNAR, TALL-1, thymic stromal lymphopoietin (TSLP), proprotein convertase subtilisin/Kexin Type 9 (PCSK9), stem cell factors, Flt-3, calcitonin gene-related peptide (CGRP), OX40L, α4β7, platelet specific (platelet glycoprotein Iib/IIIb (PAC-1), transforming growth factor beta (TFGβ), Zona pellucida sperm-binding protein 3 (ZP-3), TWEAK, platelet derived growth factor receptor alpha (PDGFRα), sclerostin, and biologically active fragments or variants of any of the foregoing.

In some embodiments, biomolecules include bispecific proteins, particularly BiTE® molecules and HLE BiTE® molecules that specifically bind CD3 in combination with CD19, CD33, EGFRvIII, MSLN, CDH19, DLL3, FLT3, CDH3, PSMA, MUC1, CLDN18.2, CD70, EpCAM, CEA, BCMA, Her2, and CD20.

The present disclosure also contemplates compositions and methods that use two or more compositions each comprising a different binding reagent as described herein. In some embodiments, a first composition comprising a CD3 peptide is used in combination with one or more compositions that comprise a binding agent that binds to a different portion or epitope of the biomolecule of interest. Here, the binding agent may be conjugated to a (different) fluorophore or otherwise modified as described herein. In various embodiments, in addition to the CD3 peptide, 1, 2, 3, 4, 5, 6, 7, 8, 9, or 10 or more binding agents are used in the methods described herein.

As used herein, a “cell” can be, in various embodiments, a eukaryotic cell, a prokaryotic cell a yeast cell, a fungal cell, an insect cell, mononuclear cells, peripheral blood mononuclear cell, bone marrow derived mononuclear cells, umbilical cord blood derived mononuclear cells, lymphocytes, monocytes, dendritic cells, macrophages, T cells, naive T cells, memory T cells, CD28⁺ cells, CD4⁺ cells, CD8⁺ cells, CD45RA⁺ cells, CD45RO⁺ cells, natural killer cells, hematopoietic stem cells, pluripotent embryonic stem cells, induced pluripotent stem cells, or plant cells. Additional exemplary cell lines include monkey kidney CV1 line transformed by SV40 (COS-7, ATCC CRL 1651); human embryonic kidney line (293 or 293 cells subcloned for growth in suspension culture, (Graham et al, J. Gen Virol. 36: 59, 1977); baby hamster kidney cells (BHK, ATCC CCL 10); mouse Sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68, 1982); MRC 5 cells or FS4 cells; mammalian myeloma cells, human liver (HuH-7) cell, CEVEC's amniocyte production (CAP) cells, hybrid kidney and B cell (HKB-11), fibrosarcoma (HT-1080) cell, human embryonic kidney (HEK) cell, human embroyonic retinal (PER.C6) cell, and Chinese hamster ovary (CHO) cells, and any other cells that are used in clinical and/or commercial manufacturing. In one embodiment, the cell or cell line can be Pichia pastoris.

As used herein, “nanofluidic chamber of a nanofluidic chip” refers to a portion or section of a nanofluidic device that is capable of isolating a single cell. By way of example, a nanofluidic chamber can be a pen associated with Berkeley Light's Beacon® technology platform as described herein. In some embodiments, the nanofluidic chip or device comprises hundreds or thousands of individual pens or chambers each capable of isolating a single cell. In some embodiments, the nanofluidic device or chip comprises 1758 chambers (or pens or wells), 3,500 chambers or 11,000 chambers.

Additional single-cell sorting and analytical platforms are also contemplated for use with reagents and materials of the present disclosure. In one embodiment, a lithographic-based microarrays or nanowell-assisted cell patterning platforms (Love et al., Nat. Biotechnol., 24(6) 703 (2006); and Ozkumur et al., Materials Views, 11(36), 4643-4650 (2015) is contemplated. Thus, in some embodiments, the nanofluidic device or chip comprises 1758 chambers, 85,000 or 250,000 chambers are contemplated herein.

As used herein, the term “expression construct” refers to a vector, a plasmid, or a linearized DNA expression sequence as routinely used in the art for the recombinant expression of proteins.

Methods for producing biomolecules are also contemplated by the present disclosure. For example, in one embodiment, a biomolecule capable of binding to a CD3 peptide is produced by collecting a single cell from a population of cells in a nanofluidic chamber of a nanofluidic chip, where the cell comprises an expression construct capable of expressing the biomolecule. After culturing the single cell under conditions that allow clonal expansion and expression and secretion of the biomolecule, a composition comprising a CD3 peptide as described herein is administered to the nanofluidic chip under conditions that allow the CD3 peptide to contact the biomolecule secreted. Next, at least one cell that secretes a biomolecule capable of binding a CD3 peptide is isolated and transferred to a vessel for culturing under conditions that allow production of the biomolecule. In various embodiments, the “vessel” may include vessel suitable for cell culture including multi-well plates, shake flasks, spin tubes, roller bottles, gas permeable culture bags, gas permeable bioreactors, gas-impermeable bioreactors, fluidized bed bioreactors, hollow fiber bioreactors, and stirred tank bioreactors of a suitable scale for the desired production level.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

### EXAMPLES

The following materials and methods were used in association with Example 1, below.

Design and Synthesis of the CD3 Peptide Fluorochrome Conjugate

The first nine n-terminal amino acids of the native human CD3e peptide amino acid sequence were used (CD3 peptide sequence used: QDGNEEMGG (SEQ ID NO: 1)). The peptide was synthesized at Metabion GmbH, Planegg, Germany and the following modifications were included: N-terminal glutamine (Q) converted to pyroglutamate, addition of cysteine to the C-terminus of the peptide, and the C-terminus amidated. The synthesized peptide was dissolved in a mixture of 85% Water, 10% Dimethylformamide DMF, 5% Acetonitrile. The Alexa Fluor C5 488 Maleimide was dissolved in DMF. The dissolved Alexa Fluor C5 488 Maleimide fluorochrome was added at a molar ratio of two fluorochromes to one peptide. Incubation was done by mixing the reaction vessel for 1 hour at room temperature in the dark. A Superdex Peptide 10/300 GL column (GE Healthcare, Freiburg, Germany) was connected to a Äkta Explorer FPLC system (GE Healthcare, Freiburg, Germany) and equilibrated with a buffer consisting of phosphate buffered saline PBS with 5% DMSO at a flow rate of 0.5 ml/min. Detection was set to online optical absorption at 280 and 490 nm wavelength. 1 ml of the reaction mixture was injected into a connected sample loop and applied to the column followed by isocratic elution using a flow of 0.5 ml/min. Eluted volume was collected in fractions of 0.5 ml size using a connected fraction collector. The first elution peak containing the peptide-fluorochrome conjugated was pooled and aliquoted.

Detection of Canonical BiTE® in Cell Culture Supernatant

100 μl of 0.2 μm filtrated mammalian cell culture samples containing expressed BiTE® constructs featuring human CD3e binding binders were mixed with 2.5 μl peptide-fluorochrome conjugate and transferred into autosampler vials. The vials were placed onto an autosampler connected to an Ultra High Performance Liquid Chromatography ACQUITY UPLC device (Waters, Eschborn, Germany) featuring a fluorescence detector. An analytical size exclusion chromatography BEH200 SEC 1.7 μm column (Waters, Eschborn, Germany) was connected to the UHPLC and equilibrated with a water-based buffer system containing 10 mM Citric Acid, 75 mM Lysine HCl, adjusted to pH 7.0. 10 μl of each prepared sample were applied to the column and eluted using isocratic elution. Detection was performed by using fluorescence detection at 490 nm excitation and 520 nm emission wavelength. Evaluation was done by comparing the elution times in a chromatogram resulting from the injection of a purified monomeric and dimeric canonical BiTE® construct as reference for elution times of the different canonical BiTE® species.

Cell Cloning Using a Berkeley Lights Beacon® Instrument

Single cell cloning methodology was implemented according to procedures described previously. (Le K, et al., Biotechnol Prog. 2018; 34(6):1438-1446; and Le K, et al., Biotechnol J. 2020; 15(1):e1900247.) In brief, stable cell lines were single cell loaded on OptoSelect chips (Design 1758, Berkeley Lights, Emeryville, CA) using the Beacon® instrument and utilizing standard Cell Line Development workflows provided by Berkeley Lights, Emeryville, CA. Cells were subjected to imaging to monitor cell growth using the integrated 4× objective and camera built in the Beacon® platform. Cells were subjected to perfusion culture using proprietary growth medium and manufacturer-recommended settings.

Spotlight® Hu3 Assay

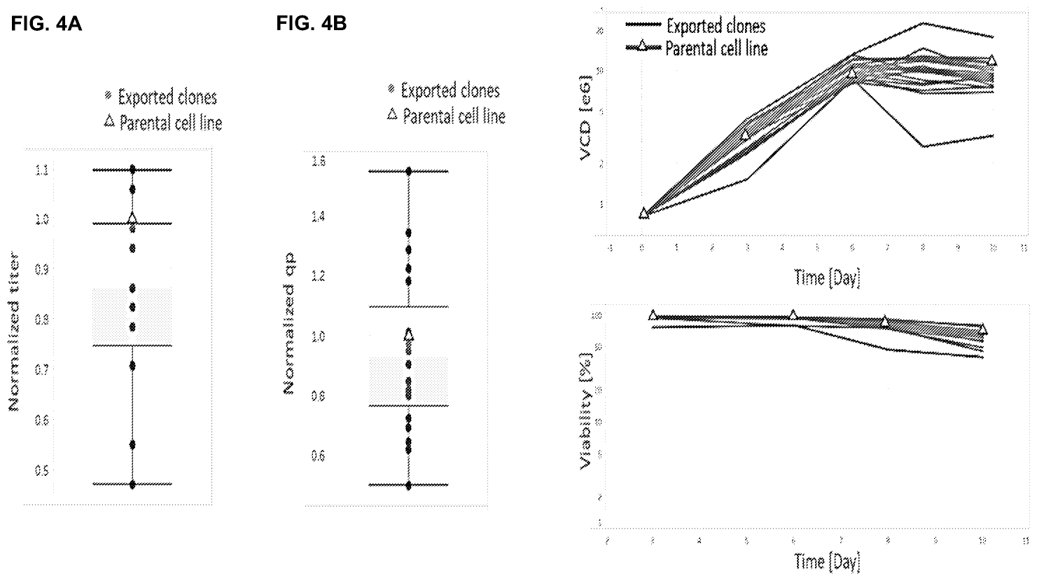

A Spotlight® Hu3 secretion assay was conducted using Spotlight® Hu3 Assay reagent provided by Berkeley Lights and used at concentration recommended by the manufacturer. The assay workflow was executed according to recommendations established by Berkeley Lights Inc. In FIG. 4B, the assay data was manually normalized to the average Au score that was calculated for empty pens by the Berkeley Lights CAS software integrated into the Beacon® platform.

CD3 Diffusion Assay

The CD3 diffusion assay peptide reagent was resuspended in DMSO at 1 mg/ml and stored protected from light at −20° C. for long term storage. For short term storage the assay reagent was further diluted to 4 μg/ml in PBS and stored at 4° C. Prior executing the diffusion gradient assay to detect BiTE® secretion on nanofluidic chip the CD3 diffusion assay reagent was diluted in cell culture medium to 0.2 μg/ml concentration, filtered through 22 μm filter, transferred to 96 well plate and incorporated on the chip. The assay reagent was perfused on the chip at 0.014 μl/sec flow rate for the duration of 45 minutes. In FIG. 4, once the equilibration step was complete the assay reagent was flushed out with 250 μl of cell culture medium at a 20/sec flow rate followed by an additional flush with the cell culture medium at the 0.1667 μl/sec flow rate for the duration of 20 mins. The chip was then imaged through TRED filter cube using 900 ms exposure time and 100% illumination settings.

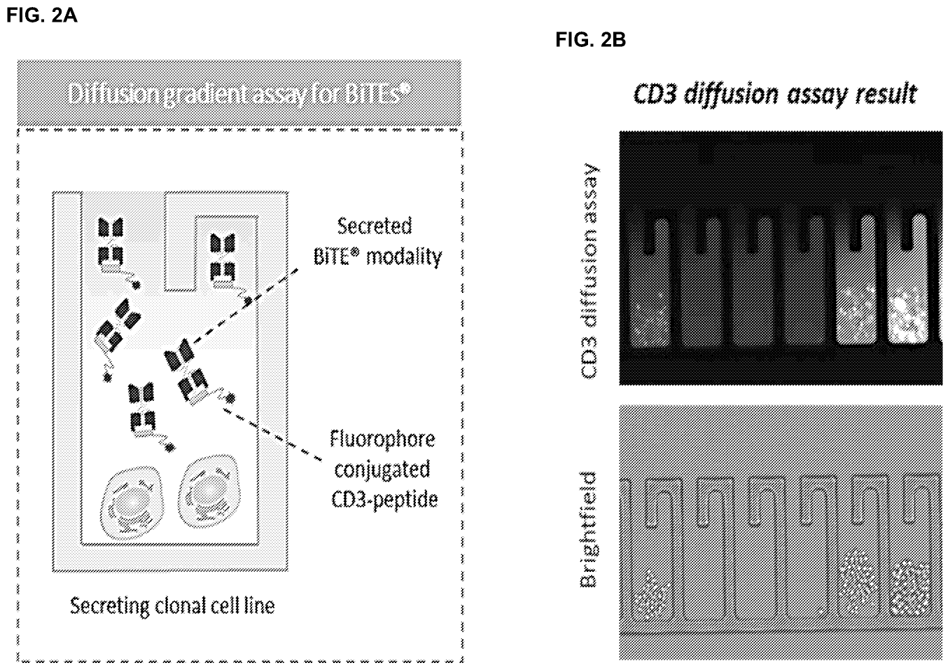

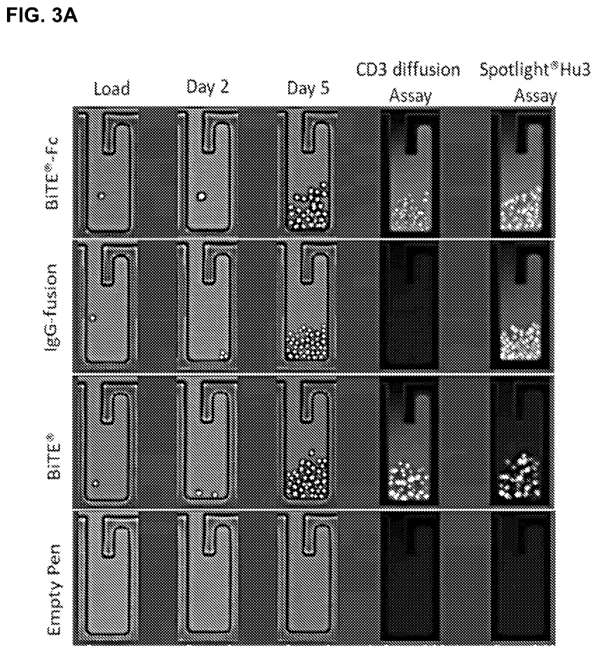

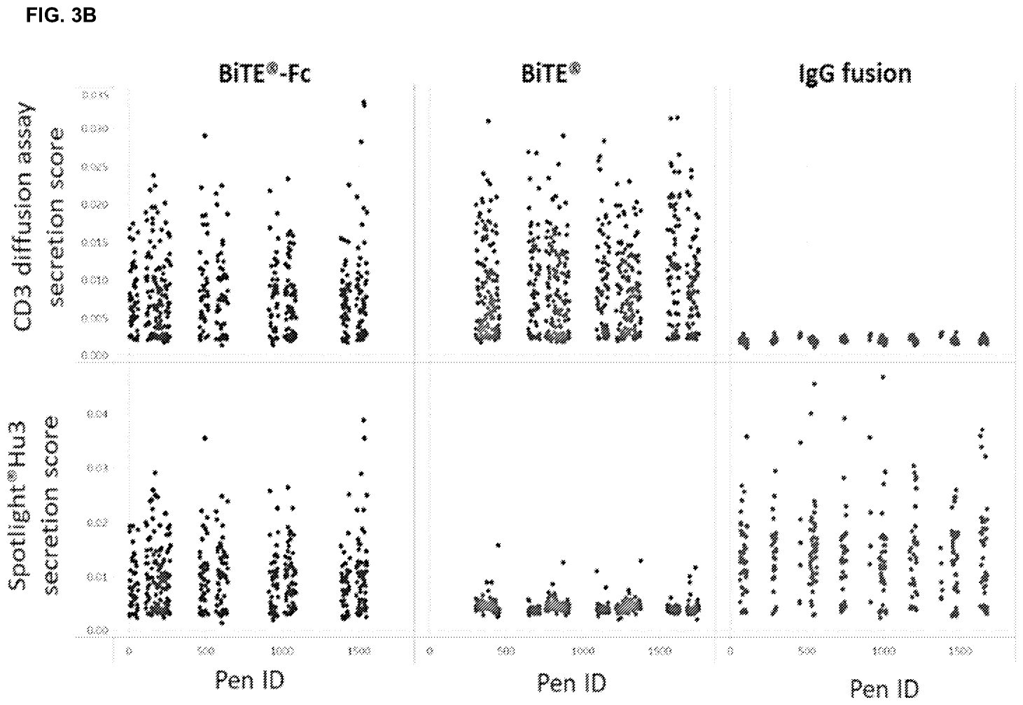

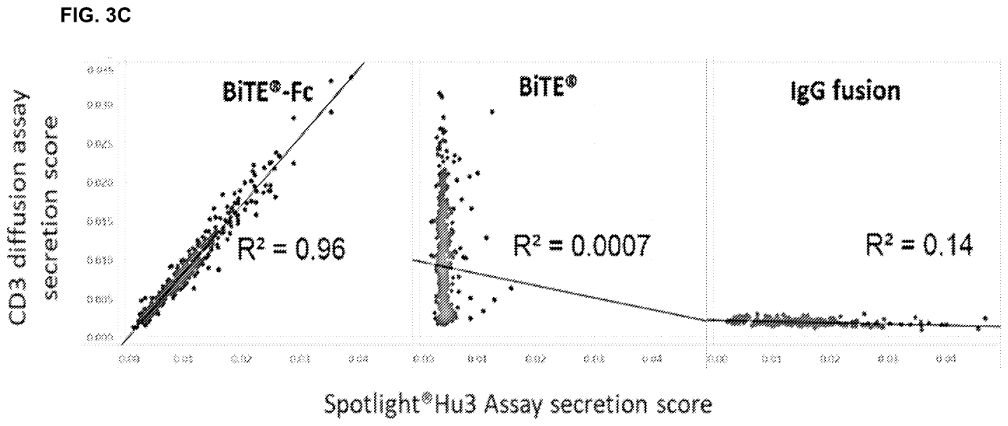

In FIG. 2, the CD3 diffusion assay reagent was used at 0.125 μg/ml final concentration. FIGS. 2 and 3, once the equilibration step was complete the assay reagent was flushed out with 500 μl of cell culture medium at 20/sec flow rate followed by additional flush with the cell culture medium at the 0.1667 μl/sec flow rate for the duration of 24 mins. The chip was then imaged through TRED filter cube using 1000 ms exposure time (FIG. 2) or 750 ms (FIG. 3) and 100% illumination settings.

Diffusion Gradient Analysis and Au Score Normalization

For CD3 diffusion assays shown in FIGS. 3 and 4, the reference assay was run on an empty chip in order to generate reference images used for normalizing the CD3 diffusion assay score against chip position effects and background fluorescence signal. The reference datasets were generated prior loading cells on a nanofluidic chip and using TRED filter cube at 900 ms exposure time and 100% illumination settings (FIG. 4) or at 750 ms exposure time and 100% illumination settings (FIG. 3). The “Background” image sequence was generated prior by incorporating CD3 diffusion assay reagent on the nanofluidic chip. The “Fluoref” image sequence was generated after the reagent was incorporated on the chip and equilibration step of the diffusion gradient assay was complete. For data in FIG. 4 the “DiffusionRef” image sequence was generated after the flush step of diffusion gradient assay was complete. The reference images were used by CAS software to normalize the CD3 diffusion assay score for each individual pen against background and chip position effects.

Dual Targeting Assay

The CD3 diffusion assay reagent was resuspended in DMSO at 1 mg/ml and stored protected from light at −20° C. for long term storage. For short term storage the assay reagent was further diluted to 4 μg/ml in PBS and stored at 4° C. Prior executing the diffusion gradient assay CD3 diffusion assay and Spotlight® Hu3 assay reagents were diluted in cell culture medium to 0.2 μg/ml and 1× concentration recommended by Berkeley Lights, respectively. The assay reagents were then filtered through 22 μm filter, transferred to 96 well plate and incorporated on the chip. The assay reagents were perfused on the chip at 0.014 μl/sec flow rate for the duration of 45 minutes. Once the equilibration step was complete the assay reagent was flushed out with 250 μl of cell culture medium at 2 μl/s is flow rate followed by additional flush with the cell culture medium at the 0.1667 μl/sec flow rate for the duration of 20 mins. The chip was then imaged through TRED filter cube using 900 ms exposure time and 100% illumination settings.

Diffusion Gradient Analysis and Au Score Normalization

The reference assay was run on an empty chip in order to generate reference images used for normalizing dual targeting assay score against chip position effects and background fluorescence signal. The reference datasets including Background”, “Fluoref” and “DiffusionRef” were generated prior loading cells on a nanofluidic chip and using TRED filter cube at 900 ms exposure time and 100% illumination settings. The pens in which bright fluorescent objects located in the measured area were identified as artifacts upon visual inspection of assay images.

10 Day Fed-Batch Production

Fed-batch experiments were conducted in 24 deep-well plates with a working volume of 3.5 mL per well, and cell suspension cultures were incubated at 36° C., 5% CO2, 85% relative humidity. Production cultures were inoculated at 8×10⁵ cells/mL target cell density, cultured for the duration of 10 days with a bolus feed at days 3, 6, and 8. In-process samples were collected on days 0, 3, 6-10 for analysis. Cell counts and viability were determined using a Vi-CellBlu cell counter (Beckman Coulter, Brea, CA). Protein titers were measured by affinity Protein A high performance liquid chromatography (HPLC) (Protein A, Waters, Milford, MA). Integrated viable cell density (IVCD) was calculated by a trapezoidal rule for VCD versus culture time. Specific productivity (Qp) was determined by titer divided by IVCD calculation.

### Example 1

### Diffusion Gradient Assay Specific for CD3 Diffusion Modalities

### A. Design and Development of the CD3 Binder Secretion Assay Reagent Mimicking CD3 Binding

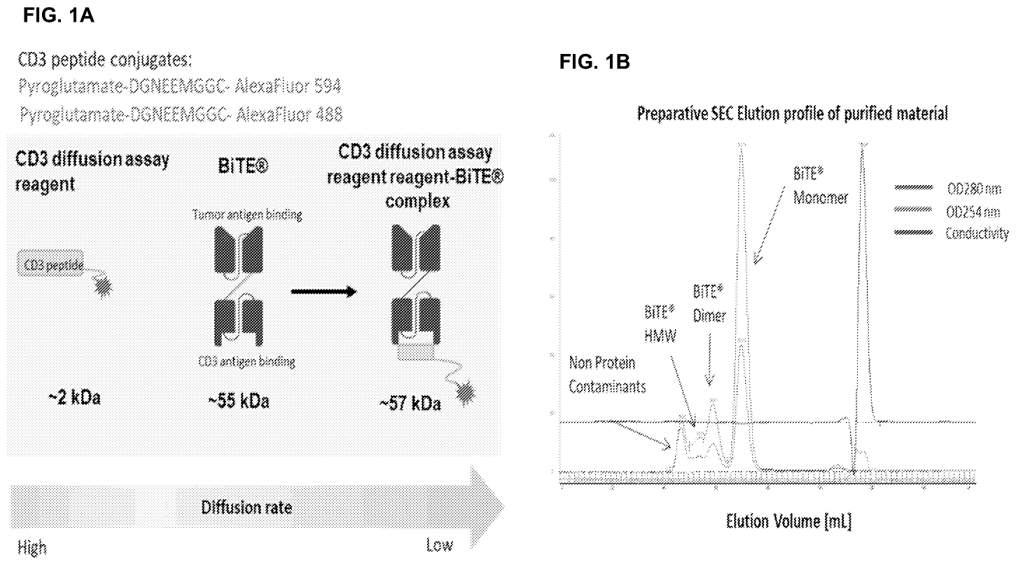

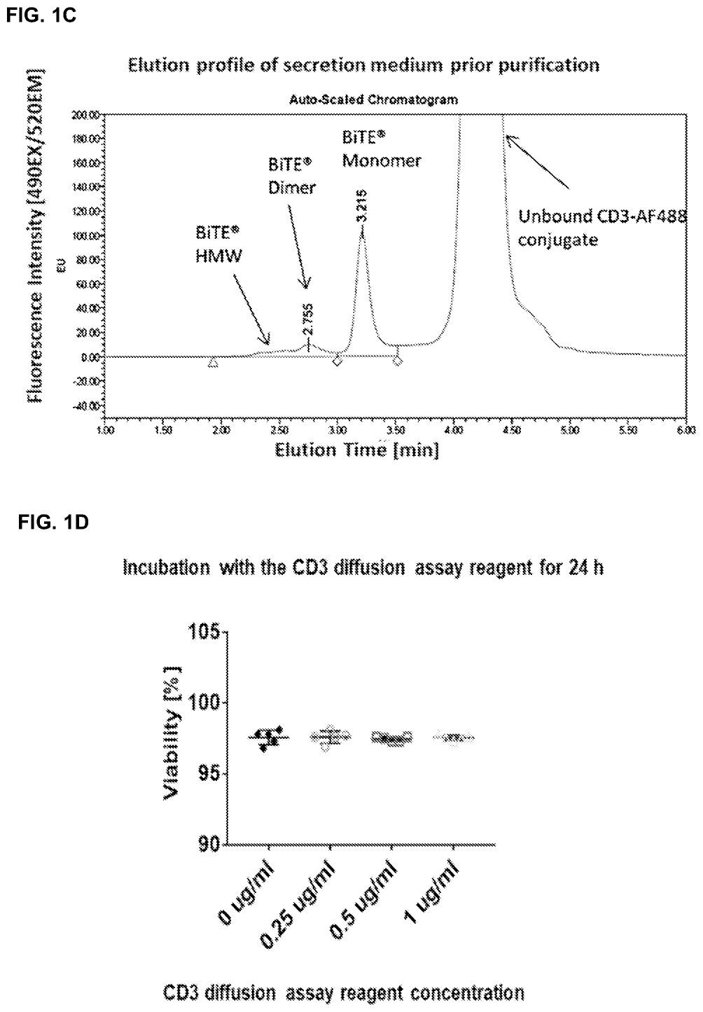

The present Example provides an assay designed to facilitate detection of CD3 binders that can make use of existing technologies including, in some embodiments, Berkeley Lights Instruments. A reagent was designed that consists of a CD3 peptide conjugated to a fluorophore that is specifically recognized by CD3 binding recombinant proteins (FIG. 1A). The specific binding of the reagent is conferred by the presence of first 9 amino acid sequence of endogenous human CD3 epsilon protein. This short peptide, due to its insolubility, proved to be difficult to synthesize. To improve peptide solubility, a cysteine residue was added to the C-terminal end, which allowed for covalent conjugation of CD3 peptide to ALEXA-FLUOR® 488 or ALEXA-FLUOR® 594 fluorophore via maleimide mediated chemistry (FIG. 1A). The the peptide was synthetically conjugated to this amino acid derivative. These modifications yielded a final peptide-conjugate of approximately 2 kDa in size with the following sequence (SEQ ID NO: 1): Pyroglutamate-DGNEEMGGC-AF594 (or AF488) (FIG. 1A).

The CD3 peptide was tested for binding to a CD3 binder, a recombinant canonical BiTE® protein secreted into the cell culture medium. Secretion medium collected from cell line expressing scFv based BiTE® recombinant protein was collected, mixed with the CD3-AF488 reagent, and subjected to capture chromatography. Both, unpurified secretion medium and purified material were subsequently analyzed by size exclusion chromatography equipped with fluorescence detector (FIG. 1B, C). This setup allowed monitoring of the elution profile of the CD3-AF488 peptide and its bound protein complexes in the context of the whole protein content present in analyzed samples. The UV absorbance (A280 nm) elution profile of purified material allowed for identification of BiTE® monomers, dimers and HMW species (FIG. 1B). The elution profile of the fluorescent CD3-AF488 peptide (254 nm) overlapped with the BiTE® elution profile demonstrating the CD3-peptide formed a complex with the BiTE® recombinant protein and allowed for detection of BiTE® monomers, dimers and aggregated species (FIG. 1B). The fluorescent elution profile detected in unpurified samples resembled the SEC profile detected in purified material demonstrating the CD3 peptide conjugate reagent can be used for detection CD3 binder secretion in cell culture medium (FIG. 1C).

To determine whether the assay reagent introduces cell toxicity, CHO cells expressing a BiTE®-Fc fusion molecule were exposed to the medium supplemented with the CD3-AF594 conjugate at concentrations ranging from 0 ug/mL to 1 μg/mL. Cells were incubated with the reagent for 24 hours, washed with medium and cell viability was analyzed. There were no observed cell viability defects in response to treatment with the CD3 assay reagent. Based on these data it was concluded that CD3 peptide conjugate was suitable for detection of CD3 binder secretion in cell culture medium and does not induce cell toxicity (FIG. 1A-D).

### B. Optimization of Diffusion Gradient Assay for CD3 Binder Secretion on Nanofluidic Chip

The ability of the CD3-AF594 peptide conjugate to be used for diffusion gradient assays to detect secretion of recombinant CD3 binders on Berkeley Light's nanofluidic chip (FIG. 2A) was assessed next. Cells expressing the BiTE® modality were loaded onto a BLI platform using a standard cell line development workflow. Single cells were deposited in Nanopens™, and subsequently cultured under perfusion for the duration of 6 days. The CD3 reagent was used in a diffusion gradient assay using optimized settings as described herein. The assay allowed identification of cell lines secreting high levels of BiTE® recombinant proteins based on visual inspection of fluorescent images collected (FIG. 2B). High fluorescent signal was detected in pens populated with cells expressing BiTE® recombinant protein, which was in contrast to the empty pens used as a negative control (FIG. 2B). These data demonstrate that the CD3 assay reagent is suitable for detection of BiTE® secretion making use of a BLI platform.

Next, the specificity of the BiTE® diffusion gradient assay was assessed. Three different cell lines expressing either a BiTE®, a BiTE®-Fc fusion or an IgG-fusion protein were used in a single cell load workflow on the BLI platform (FIG. 3A). Cells were grown for 5 days and assayed for recombinant protein secretion using Spotlight™ Hu3 and the CD3 diffusion assays. The BiTE®-Fc fusion served as a positive control in this study, as it has the capacity to bind CD3 protein as well as the affinity toward the Spotlight™ Hu3 reagent due to the presence of both a CD3 and an Fc domain. As expected, secretion of the BiTE®-Fc protein product was detected by both the Spotlight™ Hu3 and the CD3 diffusion assays (FIG. 3A,B). The secretion scores (Au scores) measured for both assays were correlated, and a R² value of 0.96 was achieved, demonstrating that the CD3 diffusion assay was able to detect the BiTE®-Fc molecules as efficiently as the commercially available Spotlight™ Hu3 assay (FIG. 3C). The CD3 diffusion assay failed to detect secretion of the IgG-fusion protein, which was in contrast to the Spotlight™ Hu3 assay, demonstrating the high specificity of the CD3 peptide reagent (FIG. 3A-C). The secretion score (Au score) measured for both the CD3 diffusion assay and the Spotlight™ Hu3 assay showed poor correlation in the case of the IgG expressing cell lines (FIG. 3C). The CD3 diffusion reagent efficiently detected secretion of the BiTE® expressing cell lines (FIG. 3A-C). These data demonstrate that the CD3 diffusion assay developed here can be applied to detect various BiTE® formats and demonstrates high specificity towards CD3 binders.

### C. Clones Selected Based on the CD3 Binder Assay Exhibits Similar Performance Compared to Clones Derived Vis Standard Methods

The performance of clones exposed to the CD3 binder assay after an export procedure (i.e., transfer of selected candidate clones from the nanofluidic chip to a 96 well plate for subsequent expansion and scaleup) was evaluated next. Clones derived from a clonal parental cell line expressing a BiTE®-Fc fusion molecule (FIG. 3) were selected and subjected to export. Nineteen clones were scaled up and assessed by a 10 day small scale fed batch experiments carried out in deep well plates. Cultures were monitored for growth, viability and final titer as well as specific productivity (qp) and were compared to the parental cell line that was used to subclone the cells on BLI chip (FIG. 3). All clones, with one exception, showed similar growth and viability profiles (FIG. 4 C,D). Titer and specific productivity achieved by the exported clones was within the range of 110-47% and 155-50%, respectively, when compared to values achieved by the parental cell line used for subcloning. All together these data demonstrated that cell lines exposed to the CD3 diffusion assay reagent during cloning demonstrate comparable performance when compared to cell lined derived via standard methods. Furthermore, the CD3 diffusion assay can be used to identify and enrich for clones showing comparable performance to cell lines selected via standard CLD workflows.

### D. Development of Dual Targeting Diffusion Gradient Assay for CD3 Binding Modalities Expressed at Low Levels

On-chip diffusion gradient assays are promising tools for detection of protein secretion at nanoscale, however; the limit of detection of for such assays presents a challenge for cell lines expressing therapeutic biologics at low levels. Utilizing an assay whereby two fluorescent probes simultaneously target non-overlapping protein domains, an increase of fluorescence signal retained in a pen could be observed.

To test this, a diffusion gradient assay was optimized for detection of BiTE®-Fc fusion proteins expressed at low levels. The BiTE®-Fc fusion protein can be recognized by both the CD3 diffusion assay and the Spotlight™ Hu3 assay due to BiTE®-Fc fusion protein's CD3 binding capacity and the presence of the Fc domain, respectively. In this embodiment, both assay regents were used. CD3 peptide and Fc assay reagents are conjugated to AF594 or TRED (Texas Red) fluorophores of overlapping spectrum, and therefore could be exited and detected simultaneously using the same fluorescent cube.

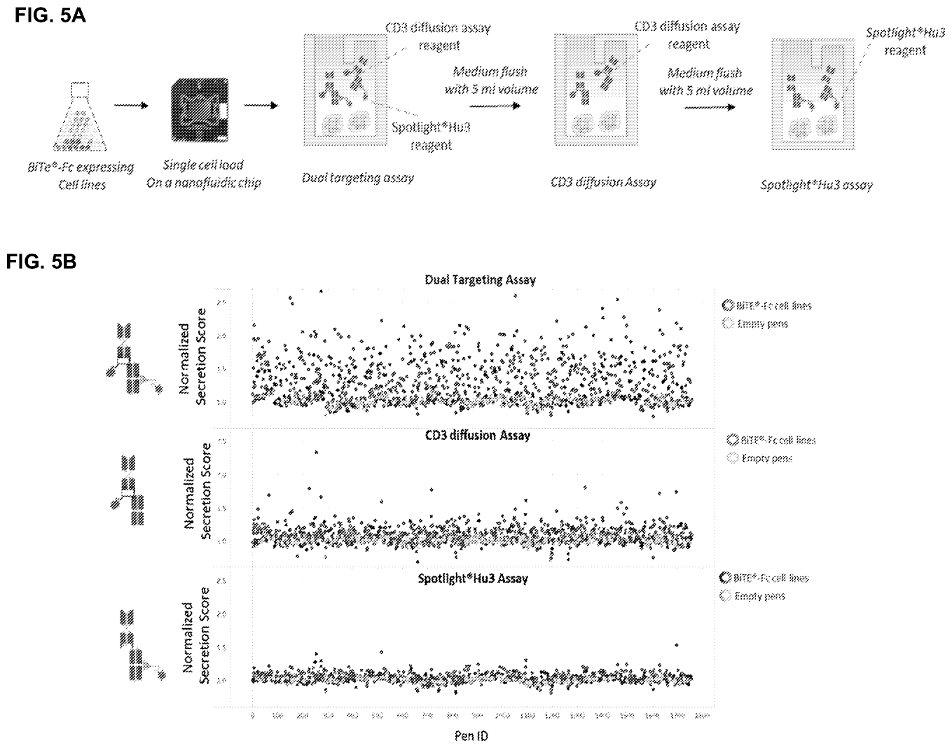

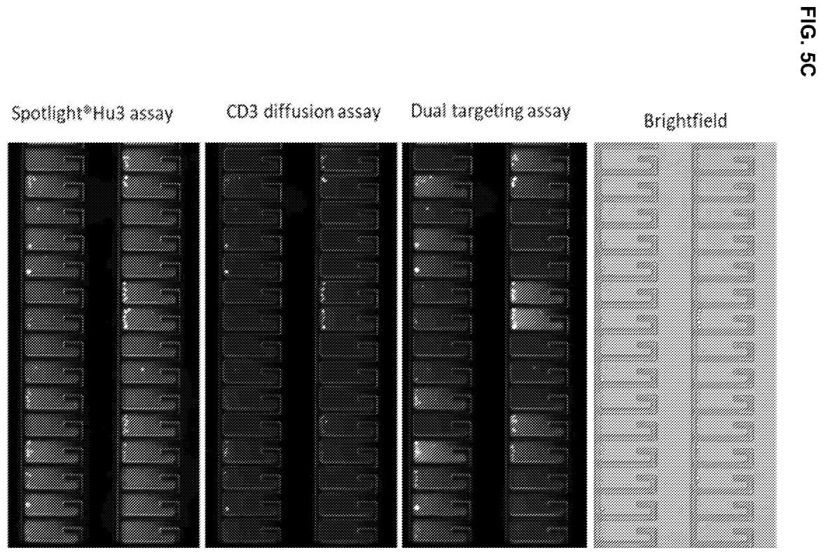

Cells expressing BiTE®-Fc protein were loaded on a BLI platform. Single cells were deposited in Nanopens™, and subsequently cultured under perfusion for the duration of 4 days. Either a dual targeting assay, where combination of both fluorescent probes was used, or a CD3 diffusion assay and Spotlight® Hu3 assay individually was then performed (FIG. 5A,B). To remove any residual fluorescence signal present in pens, the chip was flushed with 5 mls of cell culture medium in between each assay (which corresponds to approximately 1000× chip volume). Due to difference in exposure time used for image capture between Spotlight® Hu3 and the CD3 diffusion assay and dual targeting assay, the secretion score (Au score) calculated by the BLI software for each of the assays was normalized to an average Au score detected for empty pens within each dataset (FIG. 5A). This normalization step allowed for direct comparison with the secretion score for each assay executed on the chip. In case of the CD3 diffusion assay and the Spotlight® Hu3 assay, where each fluorescent probe binds to the target molecule at 1:1 ratio, the fluorescent signal detected in the pens is low. This was in contrast to the dual targeting assay where both fluorescent probes bind to a target protein product simultaneously, yielding a fluorophore:target protein ratio of 2:1. This dual recognition of a recombinant protein secreted on the chip resulted in increased fluorescence intensity detected in individual pens (FIG. 5A,B).

To determine whether the order of performing the assays and extensive flushing executed in between could have affected the protein levels detected in pens, cell lines expressing the same BiTE®-Fc fusion protein used in FIG. 4 were loaded on to a nanofluidic chip, and on Day 4 of the culture executed the Spotlight® Hu3 assay, following by 5 ml flush with medium. A dual targeting assay was performed and secretion profiles detected on the chip were analyzed. An increase in fluorescence signal while executing the dual targeting assay was detected in comparison to Spotlight® Hu3 reagent alone and highly secreting clones were easily identified.

Based on these observations it was concluded that combining two assay reagents resulted in higher sensitivity and florescence signal amplification, enabling more effective detection of BiTE®-Fc fusion proteins secreted at low levels. Furthermore, secretory phenotypes detected by utilizing both Spotlight® Hu3 and CD3 diffusion assay reagents displayed more diversity allowing for selection of clones with desired productivity profiles.

### E. Discussion

Development of cell lines expressing recombinant protein therapeutics is a resource- and time-intensive process. The Berkeley lights technology platform offers a promising solution to miniaturize cell line development activities and significantly reduces the number of clones needed to be screened in order to identify clonally derived candidate cell lines suitable for commercial manufacturing. The Beacon® platform also achieves a significant reduction in resources required to deliver high quality clonal cell lines with detailed clonality data package (Le K, et al., Biotechnol Prog. 2018; 34(6):1438-1446; and Le K, et al., Biotechnol J. 2020; 15(1):e1900247). Cell line development workflow on the Beacon® platform allows for implementation of florescent assays to aid selection of clones with desired productivity profiles. Commercially available reagents allow for detection of Fc-containing molecules which limits the ability to implement on-chip secretion assays to non-Fc modalities.

The CD3 diffusion gradient assay provided herein can detect secretion of CD3 binding modalities via a fluorescently labeled CD3 peptide. Cell lines subcloned based on secretion profiles evaluated with the CD3 diffusion assay show similar performance when compared parental cells derived from single progenitor. Furthermore, the assay reagent provided in the instant embodiment of the present disclosure is completely synthetically derived, does not induce cell toxicity, and demonstrates high specificity towards CD3 binding modality formats making it suitable for developing cell lines compatible for clinical and commercial manufacturing.

A subset of recombinant protein formats are proven to be difficult to express and their secretion occurs at low levels. In such cases detection of secreted recombinant protein product presents a challenge at the single cell or few cell level. As shown herein, combining multiple assay reagents can improve sensitivity of diffusion gradient assays used in the context of CLD workflows on the Beacon® platform and results in signal amplification.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method for isolating at least one cell from a population of cells that secretes a biomolecule capable of binding a CD3 peptide comprising the steps of:
(a) collecting a single cell from a population of cells in a nanofluidic chamber of a nanofluidic chip, wherein said cell comprises an expression construct capable of expressing said biomolecule;
(b) culturing the single cell under conditions that allow clonal expansion and expression and secretion of said biomolecule, thereby producing multiple cells from a single cell clone;
(c) administering a composition comprising said CD3 peptide to said nanofluidic chip under conditions that allow the CD3 peptide to contact said biomolecule secreted by said multiple cells of the single cell clone;
(d) detecting binding of CD3 peptide and said biomolecule; and
(e) isolating at least one cell from the multiple cells of step (b) that secretes a biomolecule capable of binding a CD3 peptide.

2. The method of claim 1 wherein the population of cells is a cell line.

3. The method of claim 1 wherein the population of cells is a mixture of two or more cell lines.

4. The method of claim 1 further comprising the step of quantifying the amount of said biomolecule secreted by said multiple cells of a single cell line.

5. The method of any of the preceding claims wherein the cell is selected from the group consisting of a mammalian cell, an insect cell, a bacterial cell, a eukaryotic cell, a plant cell, a yeast cell and a fungal cell.

6. The method of claim 5 wherein the cell is selected from the group consisting of a Chinese hamster ovary (CHO) cell, human embryonic kidney (HEK) cell, murine myeloma (NS0, Sp2/0) cell, baby hamster kidney (BHK) cell, human embryonic kidney (293) cell, fibrosarcoma (HT-1080) cell, human embryonic retinal (PER.C6) cell, hybrid kidney and B cell (HKB-11), CEVEC's amniocyte production (CAP) cells, and human liver (HuH-7) cell.

7. The method of any of the preceding claims wherein said biomolecule comprises a polypeptide.

8. The method of claim 7 wherein said polypeptide is recombinant.

9. The method of any one of claims 7-8 wherein said polypeptide is selected from the group consisting of an antibody, a peptibody, a multispecific protein, a bispecific protein, a bi-specific T cell engager, a half-life extended bi-specific T cell engager, and biologically active fragments, analogs and derivatives thereof.

10. The method of any of the preceding claims wherein the biomolecule is a BiTE and is also capable of binding a target molecule selected from the group consisting of CD33, EGFRvIII, MSLN, CDH19, DLL3, CD19, FLT3, CDH3, BCMA, PSMA, MUC17, CLDN18.2, EpCAM, CEA, Her2, CD20 and CD70.

11. The method of any of the preceding claims wherein said CD3 peptide comprises 5, 6, 7, 8, 9, 10, 11, 12, 13, 14, 15, 16, 17, 18, 19 or 20 amino acids.

12. The method of any of the preceding claims wherein said CD3 peptide comprises 10 amino acids.

13. The method of claim 12 wherein said CD3 peptide comprises the amino acid sequence Pyroglutamate-DGNEEMGGC (SEQ ID NO: 1).

14. The method of any of the preceding claims wherein said CD3 peptide is a CD3 peptide-conjugate comprising at least one modification.

15. The method of claim 14 wherein said modification comprises attachment of a detection moiety selected wherein said detection moiety is a fluorophore.

16. The method of claim 15 wherein said fluorophore is selected from the group consisting of AF594 and AF488.

17. The method of claim 16 wherein said AF594 is attached to the C-terminal cysteine of the sequence Pyroglutamate-DGNEEMGGC (SEQ ID NO: 1) using a maleimide linker.

18. The method of any of the preceding claims wherein said expression construct is selected from the group consisting of a vector, a plasmid, and a linearized DNA expression sequence.

19. The method of any of the preceding claims wherein said method is completed in 14 days or less.

20. The method of any of the preceding claims wherein said nanofluidic chip comprises between 1,000 and 2,000 nanofluidic chambers.

21. The method of claim 20 wherein said nanofluidic chip comprises 1758 nanofluidic chambers.

22. A method for isolating at least one cell from a population of cells that secretes a bi-specific T cell engager capable of binding a CD3 peptide comprising the steps of:
(a) collecting a single cell in a nanofluidic chamber of a nanofluidic chip, wherein said cell comprises an expression construct capable of expressing said bi-specific T cell engager;
(b) culturing the single cell under conditions that allow clonal expansion and expression and secretion of said bi-specific T cell engager, thereby producing multiple cells from a single cell clone;
(c) administering a composition comprising said CD3 peptide to said nanofluidic chip under conditions that allow the CD3 peptide to contact said bi-specific T cell engager secreted by said multiple cells of the single cell line;
(d) detecting binding of CD3 peptide and said bi-specific T cell engager; and
(e) isolating at least one cell from the multiple cells of step (b) that secretes a bi-specific T cell engager capable of binding a CD3 peptide;
wherein said CD3 peptide is a CD3 peptide conjugate and comprises the amino acid sequence Pyroglutamate-DGNEEMGGC (SEQ ID NO: 1), wherein AF594 is attached to the C-terminal cysteine of said sequence.

23. A method of producing a biomolecule capable of binding a CD3 peptide comprising the steps of:
(a) collecting a single cell from a population of cells in a nanofluidic chamber of a nanofluidic chip, wherein said cell comprises an expression construct capable of expressing said biomolecule;
(b) culturing the single cell under conditions that allow clonal expansion and expression and secretion of said biomolecule, thereby producing multiple cells from a single cell clone;
(c) administering a composition comprising said CD3 peptide to said nanofluidic chip under conditions that allow the CD3 peptide to contact said biomolecule secreted by said multiple cells of the single cell line;
(d) detecting binding of CD3 peptide and said biomolecule;
(e) isolating at least one cell from the multiple cells of step (b) that secretes a biomolecule capable of binding a CD3 peptide; and
(f) transferring said at least one cell from the cell line to a vessel and culturing said cell under conditions that allow production of the biomolecule.

24. A method for isolating at least one cell from a population of cells that secretes a biomolecule capable of binding a CD3 peptide comprising the steps of:
(a) collecting a single cell from a population of cells in a nanofluidic chamber of a nanofluidic chip, wherein said cell comprises an expression construct capable of expressing said biomolecule;
(b) culturing the single cell under conditions that allow clonal expansion and expression and secretion of said biomolecule, thereby producing multiple cells from a single cell clone;
(c) administering a first composition comprising said CD3 peptide and a second composition comprising a biomolecule binding-reagent to said nanofluidic chip under conditions that allow the CD3 peptide to contact said biomolecule secreted by said multiple cells of the single cell line;
(d) detecting binding of the CD3 peptide and the biomolecule binding agent and said biomolecule; and
(e) isolating at least one cell from the multiple cells of step (b) that secretes a biomolecule capable of binding a CD3 peptide and, optionally, the biomolecule binding agent.
